(19) United States
(12) Patent Application Publication
(10) Pub. No.: US 2011/0147257 A1
(43) Pub. Date: Jun. 23, 2011

(54) PACKAGE FOR ABSORBENT ARTICLES

(75) Inventors: Tsuyoshi Takahashi, Kagawa (JP); Kayoko Sato, Kagawa (JP); Mika Fukushima, Kagawa (JP); Yuko Suda, Kagawa (JP)

(73) Assignee: UNI-CHARM CORPORATION, Ehime (JP)

(21) Appl. No.: 12/995,637

(22) PCT Filed: Apr. 20, 2009
(86) PCT No.: PCT/JP2009/057867

§ 371 Date: Feb. 25, 2011

(30) Foreign Application Priority Data
Jun. 2, 2008 (JP) 2008-145163

(51) Int. Cl. A61F 13/84

(52) U.S. Cl. 206/459.5

(57) ABSTRACT

The present invention aims to provide a package for absorbent articles allows the consumers to recognize at a glance principal functional information of the article displayed on the front surface of the package and thereby to be adequately informed of these principal functions. Two or more function displays on the front surface of the article package may be divided into a main-function display element and sub-function display elements wherein the main-function display element is displayed to be accentuated in comparison with the respective sub-function display elements.

### TECHNICAL FIELD

The present invention relates to packages for absorbent articles and more particularly to packages for disposable diapers particularly tailored for babies (infants) provided on a front surface of the package with display elements allowing consumers to obtain various kinds of functional information on each packaged article.

### RELATED ART

Conventionally, on a front surface of a package for baby's disposable diapers, in addition to a trade name (article name), a trademark, an appearance of the diaper, a figure of wearer, functions of this article (diaper) are displayed and, on the rear surface of the package, these functions are displayed by graphic arts and letters in more detail.

As one example of prior arts, a known package for baby's disposable diapers illustrated by FIGS. 4 and 5 of the accompanying drawings is cited here. It should be appreciated here that FIGS. 4 and 5 are partially eliminated diagrams schematically illustrating front and rear surfaces of the package. In this package 100, a trademark 102, a FIG. 104 of a baby lying on his or her back and wearing a diaper 103, letters 105 identifying the diaper 103 as the diaper for newborns and graphic arts displaying respective functions A through D of the diaper 103 in the form of puzzle pieces 106a through 106d separated by color are displayed on the front surface. On the rear surface 101b, a developed partial diagram of the diaper 103 is displayed and regions in this developed diagram having the respective functions A through ID are displayed in association with the respective graphic arts 106a through 106d via lead tracks and letters 107a through 107d are displayed adjacent the respective graphic arts 106a through 106d, respectively, for further explanation of the respective functions A through D.
[Non-patent Document 1] Package for disposable diaper of prior art as illustrated by FIGS. 4 and 5 of the accompanying drawings.

### DISCLOSURE OF THE INVENTION

### Problem to be Solved by the Invention

However, display of the respective functions of the diaper in the above-cited prior art is nothing but enumeration even though the display is separated by color or shape. Even if the consumer (e.g., mother) takes the detailed explanation of the respective functions displayed on the rear surface of the package, the display is not arranged to suggest which is the most important function of the diaper is to be worn by the wearer. In consequence, the consumer will select and buy the diaper not on the basis of firm recognition of this diaper's functions and it is impossible for the consumer to select and buy the diaper considered to have the principal functions required or preferable for the wearer.

Furthermore, the package for commercially available disposable diapers inclusive of the package for prior art is usually provided with the growth stage display of the wearer, specifically, the display (See, e.g., the letters 105) indicating that the packaged diaper is for the wearer at a predetermined age in month or younger.

However, the display of the respective function is not related with the growth stage display. In other words, the respective functions are not displayed to suggest one or more functions are necessary or preferable for the wearer in the stage indicated by the growth stage display. Consequently, it is impossible for the consumer to select and buy the diaper considered to have such necessary or preferable functions.

In view of the problems as have been described above, it is one principal object of the present invention is to provide a package for absorbent articles wherein a display of two or more functions of the article is divided into a main-function display element and sub-function display elements and the main-function display element is formed to be accentuated in comparison with the sub-function display elements so that consumers can recognize at a glance at least the main-function display element and the consumers can be adequately informed of these functions.

In view of the problems as have been described above, it is another principal object of the present invention is to provide a package for absorbent articles wherein, in addition to the features described just above, the main- and sub-function display elements are displayed in correspondence relationship with the wearer's growth stage display element displayed on the front surface of the package so that the consumer can recognize the main- and sub-function display elements in correspondence relationship with the growth stage display element and the consumer can be adequately informed of these functions.

### Measure to Solve the Problem

The objects set forth above are achieved, according to the present invention, by an improvement in package for absorbent articles provided on a front surface with at least a trade name or a trademark and functions of each of the packaged article displayed by letters and/or graphic arts. The present invention is characterized as will be described below.

A display of the functions includes one main-function display element and two or more sub-function display elements. The main- and sub-function display elements are arranged in appropriate regions of a front surface of the package closely adjacent one to another to form one group. The main-function display element occupies a display region larger than respective display regions of the two or more sub-function display elements. The main-function display element includes graphic arts and letters. The sub-function display elements include graphic arts and/or letters.

The present invention includes preferred embodiments as follow.

The main- and sub-function display elements respectively include outlines adapted to encircle the graphic arts and/or letters and the outline for the main-function display element has a size larger than respective the outlines for the two or more sub-function display elements.

The graphic arts and letters serve to display the functions.

The graphic arts display the article substantially wholly as viewed from its inside and outside and regions having the functions are indicated inside and/or outside the graphic arts by at least one of an illustration, a mark and a color.

The package further includes a wearer's growth stage display element arranged in an appropriate region of the front surface of the package and the growth stage display element includes graphic arts and/or letters.

The growth stage display element is arranged in the vicinity of the one group of the main- and sub-function display elements.

The main- and sub-function display elements display functions of the article tailored for the wearer in the stage displayed by the growth stage.

The graphic arts in the growth stage display element include the figure of the wearer and/or a character.

### Effect of the Invention

In the package for absorbent articles according to the present invention, one main-function display element and two or more sub-function display elements are arranged closely adjacent one to another to form one group on the front surface of the package. Obviously, the present invention does not exclude displaying various functions of the article on the rear surface of the package and anticipation that the consumers check such information displayed on the rear surface of the package. However, the present invention allows the consumers to recognize at a glance various functions of the article at least from the main- and sub-function display elements displayed on the front surface of the package and thereby to be informed of these functions, particularly the main-functions. Thus the consumers can adequately recognize this functional information and selectively buy the article.

The front surface of the package additionally includes the growth stage display element displaying the growth stage of the wearer adapted to wear the packaged article. The main- and sub-function display elements may be correlated with the growth stage display element to inform the consumers having babies in the growth stage displayed on the front surface of the package for the main- and sub-function display elements corresponding to the growth state display element. In this way, the consumers can adequately recognize this functional information and selectively buy the article.

### BRIEF DESCRIPTION OF THE DRAWINGS

FIG. 1 is a plan view schematically illustrating a front surface of the package for the diaper as a typical example of absorbent articles tailored for newborn babies of predetermined month age or less and having a body weight in a range of a birth weight to 5 kg.

FIG. 2 is a view similar to FIG. 1, schematically illustrating the front surface of the package for the diapers tailored for wearers having become older than the stage displayed in a growth stage display element (predetermined age in month) and each having a body weight in a range of 4 to 8 kg so that the wearers can move their bodies rather vividly.

FIG. 3 is a view similar to FIG. 1, schematically illustrating the front surface of the package for the diapers tailored for the wearers having further become older in month age and each having a body weight in a range of 6 to 11 kg so that the wearers can sit up.

FIG. 4 is a view similar to FIG. 1, schematically illustrating the front surface of the package for baby's disposable diapers of a prior art.

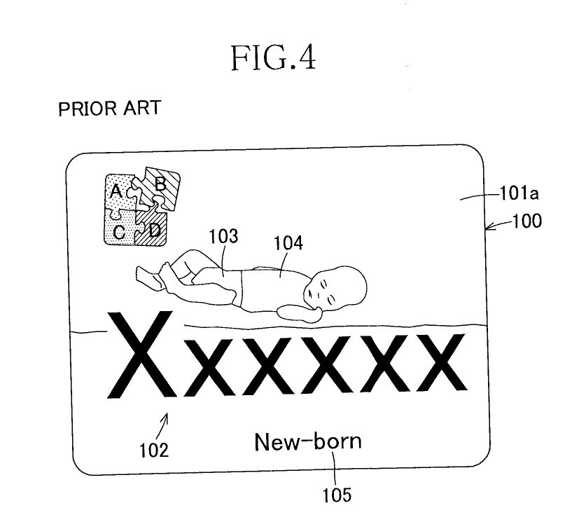

FIG. 5 is a plan view schematically illustrating the rear surface of the package for the prior art of FIG. 4.

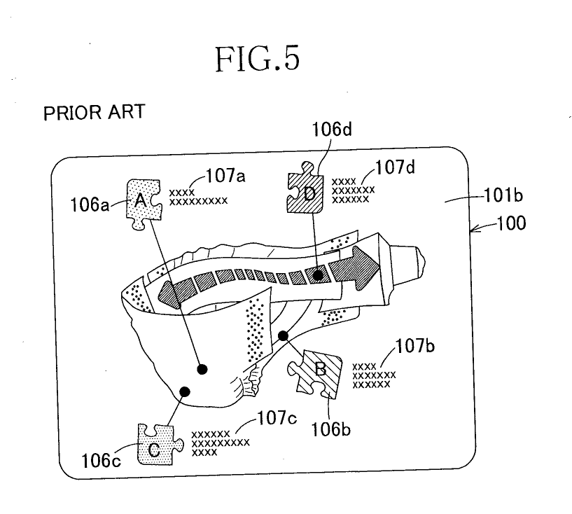

### IDENTIFICATION OF REFERENCE NUMERALS USED IN THE DRAWINGS

10 package
11 front surface
12 trademark
13 illustration of diaper
14 figure of wearer
17 main function display element
18 sub-function display element
19 sub-function display element
20 graphic art element
21 graphic art
22 circular outline
23 letters
24 circular outline
25 letters
26 circular outline
27 letters
28 letters
29 letters
30 letters
31 imaginary baby mark
32 growing stage display element

### DETAILED DESCRIPTION OF THE PREFERRED EMBODIMENTS

Preferred embodiments of the present invention will be described with reference to the accompanying drawings.

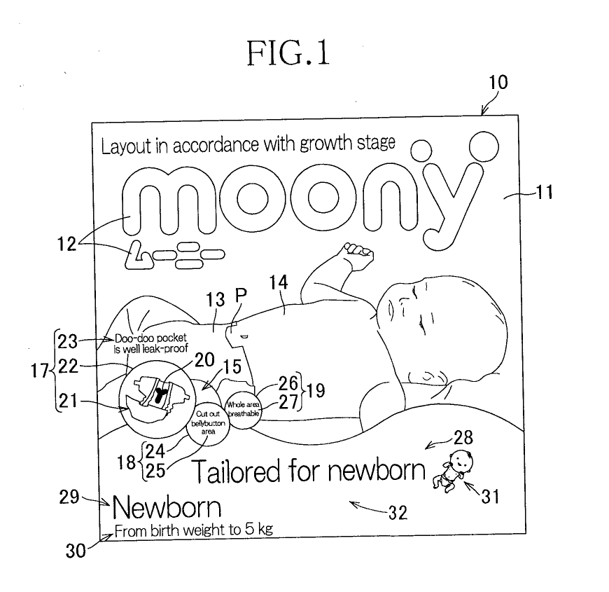

FIG. 1 is a plan view schematically illustrating a front surface of the package for the diapers as a typical example of the absorbent articles tailored for newborn babies of predetermined month age or less and each having a body weight in a range of a birth weight to 5 kg. The package 10 having on a front surface 11, a trademark 12, a FIG. 14 of a wearer of the diaper 13, a function display 15 of the diaper 13 and a display element 31 of a wearer's growth stage.

The function display 15 includes one main function display element 17 and two sub-function display elements 18, 19. The main and sub-function display elements 17, 18, 19 are arranged closely one to another to form one group.

The main function element 17 includes a developed perspective view 21 illustrating the inner side of the diaper having a colored graphic art 20 symbolically showing feces retaining pocket formed inside the diaper, a circular outline 22 encircling the graphic art 21 and letters 23 written above and outside the circular outline 22 representing “doo-doo pocket well prevents doo-doo from leaking out”.

The sub-function display element 18 includes a circular outline 24 smaller than the circular outline 22 for the main function display element 17 and letters 25 written within this circular outline 24 and representing “cut bellybutton area”. Similarly, the other sub-function display element 19 includes a small circular outline 26 and letters 27 written within this circular outline 24 and representing “whole area breathable”.

As will be apparent from an occupancy ratio the circular outline 22 for the main function display element 17 and the circular outline 26 for the sub-function display elements 18 19 on the front surface 11 of the package 10, a display area of the main function display element 17 is larger than respective display areas of the sub-function display elements 18, 19.

Consequently, both the main function display element 17 and the sub-function display elements 18, 19 well show up and particularly the main function display element 17 stands out, assuring that the consumer can be reliably informed of these functions. To make it further sure, these main- and sub-function display elements 17, 18, 19, the circular outlines 22, 24, 26 and the letters 23, 25, 27 may be respectively separated by color for emphatic effect.

The circular outlines 22, 24, 26 may be replaced by the outlines having other curve geometry such as oblong, heart-shaped or cloverleaf-shaped geometry, or angulated geometry such as quadrangular, triangular or rhombic geometry. It is also possible without departing from the scope of the invention to combine the curve geometry with the angulated geometry.

The letters 23 defining the main-function display element 17 may be arranged inside the circular outline 22. The letters 25, 27 defining the respective sub-function display elements 18, 19 also may be arranged outside the respective circular outlines 24, 26 so far as these letters are adjacent to the respective circular outlines.

While not illustrated, it is possible for the circular outlines 24, 26 encircling the sub-function display elements 18, 19, respectively, without departing from the scope of the invention to contain therein symbolic graphic arts associated with the letters “cut bellybutton area”, “whole area breathable” 25, 27, respectively, in the place of or together with these letters 25, 27 (See the graphic art element 20 complementing the main-function display element 20). Regarding the sub-function display elements 18, 19, one or more sub-function display element(s) may be added, if desired.

The symbolic graphic art element 20 included in the main-function display element 17 as well as any similar symbolic graphic art element added, if desired, may be accentuated by color or the other marks such as arrows upon which the consumers may be impressed.

As the FIG. 14 of the wearer, a newborn baby at a predetermined age in month or younger lying on his or her back and sleeping is displayed substantially in the central region on the front surface 11 of the package 10 in a relatively large size compared to the other displays. At least one of this FIG. 14, the letters 28 “tailored for newborn”, the letters 30 “from birth weight to 5 kg”, and the imaginary baby mark 31 lying face up assures the consumer to understand that the diapers within the package 10 is for newborn babies at a predetermined age in month or younger.

According to the present invention, the FIG. 14 of the wearer, the letters 28, 29, 30 and the imaginary baby mark 31, particularly the FIG. 14 and the letters 28 are adapted to inform the consumers of the fact that this baby is on the growth stage at a predetermined age in month or younger. Such figure and/or the letters may be defined as the growth stage display element 32.

This growth stage display element 32 is displayed in the vicinity of a group of the main- and sub-function display elements 17, 18, 19. for this reason, these display elements 17, 18, 19 readily come in sight of the consumer at a glance and therefore the consumer can visually recognize correspondence relationship between the main- and sub-function display elements 17, 18, 19 and the growth stage display element 32.

While the letters 23, 24 of the main- and sub-function display elements 17, 18 are represented in relatively friendly colloquial style, it is possible to represent these letters 23, 24 in the literary style or the styles containing literary style so far as the representation same as or substantially same as the representation of the letters written in colloquial style are informed to the consumers.

An aspect of the present invention which should be kept in mind is a correspondence relationship between the main- and sub-function display elements 17, 18, 19 and the growth stage display element 32.

Referring to FIG. 1, of the main- and sub-function display elements 17, 18, 19, at least the functions represented by the graphic art element 20 or the letters 23 of the main-function display element 17 are required or preferable for the diaper adapted for a newborn baby at a predetermined age in month or younger. Such newborn baby at a predetermined age in month or younger normally excretes loose passage which would otherwise flow and disperse on the topsheet of the diaper and soil the wearer's skin, causing so-called skin trouble. To prevent such loose passage from flowing and dispersing, it is required or preferable to provide the diaper with the feces retaining pocket (doo-doo pocket). In many cases, the newborn baby at such age in month is not long after his or her naval cord has been cut away. Considering this, it is undesirable that the component material of the diaper might come in direct contact with the baby's bellybutton. To avoid this direct contact, it is required or preferable to cut away a region of the diaper supposed to come in direct contact with the bellybutton. (See the region designated by referential mark P in the FIG. 14 of FIG. 1).

In this way, the consumer is surely informed of the fact that the diaper adapted to be worn by the baby in such growth stage is provided with the functions displayed by the main- and sub-function display elements 17, 18.

Breathability assured over whole area displayed by the sub-function display element 19 is required or preferable for the baby whose age in month has further increased and this sub-function display element 19 also is useful functional information for the consumers.

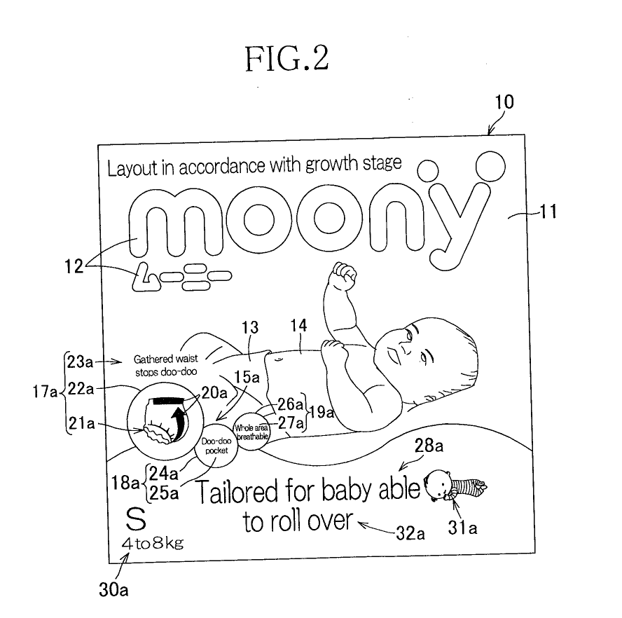

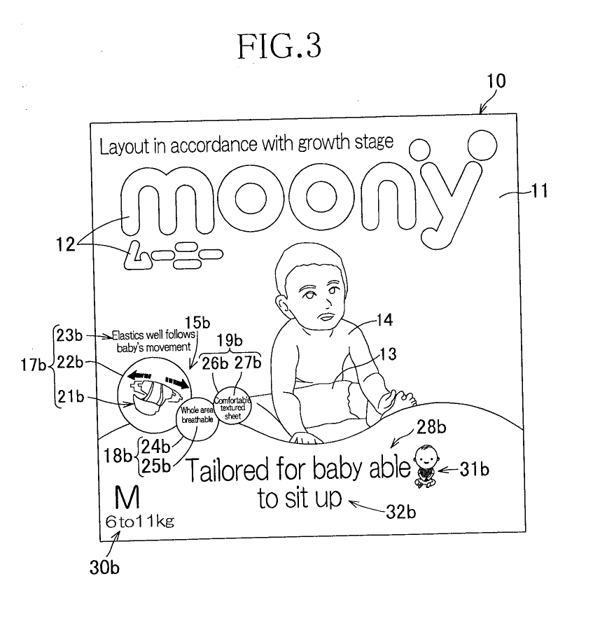

FIG. 2 is a plan view schematically illustrating the front surface of the package for the diapers tailored for the wearers each having become older than the stage displayed in the growth stage display element 32 (predetermined age in month or younger) and having a body weight in a range of 4 to 8 kg so that the wearer can move his or her body rather vividly. FIG. 3 a plan view schematically illustrating the front surface of the package for the diaper tailored for the wearers each having further become older in month age and having a body weight in a range of 6 to 11 kg so that the wearer can sit up.

The packages 10 illustrated by FIGS. 2 and 3 are similar to the package 10 shown by FIG. 1 in construction and purpose except the expressive manner of the main- and sub-function display elements 17, 18, 19 and the growth stage display element 32. Therefore, the similar elements, letters, figure and graphic arts are designated by similar reference numerals or marks and repetitive description thereof will be avoided.

A function display 15a on the package 10 shown by FIG. 2 includes one main-function display element 17a and two sub-function display elements 18a, 19a. The main- and sub-function display elements 17a, 18a, 19a are arranged adjacent one to another to form one group.

The main-function display element 17a includes a developed perspective view 21a of the diaper as viewed from outside in which a colored graphic art element 20a symbolically illustrating gathered waist of the diaper and indicated by an arrow, and letters 23a representing “fully gathered waist stops doo-doo” arranged above and outside a circular outline 22a encircling the perspective view 21a.

The one sub-function display element 18a includes a circular outline 24a smaller than the circular outline 22a for the main-function display element 17a and letters 25a arranged inside the circular outline 24a and representing “doo-doo pocket”. In like wise, the other sub-function display element 19a includes a relatively small circular outline 26a and letters 27a arranged inside the circular outline 26a and representing “whole area breathable”.

A growth stage display element 32a includes letters 28a representing “tailored for baby able to roll over”, letters 30a of “S 4 to 8 kg” and in imaginary baby mark 31a representing a baby lying face down.

A function display 15b on the package 10 illustrated by FIG. 3 includes one main-function display element 17b and two sub-function display elements 18b, 19b. The main- and sub-function display elements 17b, 18b, 19b are arranged adjacent one to another to form one group.

The main-function display element 17b includes a developed perspective view 21b of the diaper as viewed from inside, a circular outline 22b encircling the perspective view 21b and letters 23b arranged above and outside the circular outline 22b and representing “elastics well follows movement of the wearer”.

The one sub-function display element 18b includes a circular outline 24b smaller than the circular outline 22b for the main-function display element 17b and letters 25b arranged inside the circular outline 24b and representing “whole area breathable”. In like wise, the other sub-function display element 19b includes a relatively small circular outline 26b and letters 27b arranged inside the circular outline 26b and representing “comfortable textured sheet”.

A growth stage display element 32b includes letters 28b representing “tailored for baby able to sit up”, letters 30b representing “M 6 to 11 kg” and an imaginary baby mark 31b representing a baby sitting up.

The purpose of the main- and sub-function display elements 17a, 18a, 19a; 17b, 18b, 19b and the growth stage display elements 32a, 32b on the packages 10 illustrated by FIGS. 2 and 3, respectively, is the same as the purpose of such display elements on the package 10 illustrated by FIG. 1. In other words, the main- and sub-function display elements 17a, 18a, 19a; 17b, 18b, 19b on the packages 10 illustrated by FIGS. 2 and 3 are in correspondence relationship with the growth stage display elements 32a, 32b, respectively. Expression “in correspondence relationship” used here means the relationship which has been described above with respect to FIG. 1.

It should be understood here that the present invention relates to displays on the front surface of the package and the rear surface of the package 10 is not illustrated in FIGS. 1 through 3. Usually the other functions of the diaper are displayed in details and the present invention neither exclude nor restrict display of various kinds of information such as functions of the diaper on the rear surface of the package.

1. A package for absorbent articles provided on a front surface with at least a trade name or a trademark and functions of each of said packaged article displayed by letters and/or graphic arts, said package for absorbent articles;
wherein a display of said functions includes one main-function display element and two or more sub-function display elements;
said main- and sub-function display elements are arranged in appropriate regions of a front surface of said package closely adjacent one to another to form one group;
said main-function display element occupies a display region larger than respective display regions of said two or more sub-function display elements;
said main-function display element includes graphic arts and letters; and
said sub-function display elements include graphic arts and/or letters.

2. The package for absorbent articles defined by claim 1 wherein said main- and sub-function display elements respectively include outlines adapted to encircle said graphic arts and/or letters and said outline for said main-function display element has a size larger than respective said outlines for said two or more sub-function display elements.

3. The package for absorbent articles defined by claim 1 wherein said graphic arts and letters serve to display said functions.

4. The package for absorbent articles defined by claim 1, wherein said graphic arts display said article substantially wholly as viewed from its inside and outside and regions having said functions are indicated inside and/or outside said graphic arts by at least one of an illustration, a mark and a color.

5. The package for absorbent articles defined by claim 1, wherein said package further includes a wearer's growth stage display element arranged in an appropriate region of the front surface of the package and said growth stage display element includes graphic arts and/or letters.

6. The package for absorbent articles defined by claim 5 wherein said growth stage display element is arranged in the vicinity of said one group of said main- and sub-function display elements.

7. The package for absorbent articles defined by claim 5 wherein said main- and sub-function display elements display functions of the article tailored for the wearer in the stage displayed by said growth stage.

8. The package for absorbent articles defined by claim 5, wherein said graphic arts in said growth stage display element include the figure of the wearer and/or a character.
